(19) United States
(12) Patent Application Publication
(10) Pub. No.: US 2021/0113332 A1
(43) Pub. Date: Apr. 22, 2021

(54) INVERTIBLE VALVE SUPPORT FRAME FOR USE WITH PROSTHETIC HEART VALVE APPARATUS

(71) Applicant: TRULEAF MEDICAL LTD., Caesarea (IL)

(72) Inventors: Netanel BENICHOU, Hof Carmel (IL); Benjamin SPENSER, Hof Carmel (IL)

(73) Assignee: TRULEAF MEDICAL LTD., Caesarea (IL)

(21) Appl. No.: 16/498,099

(22) PCT Filed: Mar. 1, 2018
(86) PCT No.: PCT/IL2018/050230

§ 371 Date: Sep. 26, 2019

Related U.S. Application Data
(60) Provisional application No. 62/476,979, filed on Mar. 27, 2017.

(51) Int. Cl. A61F 2/24

(52) U.S. Cl. CPC A61F 2/2433; A61F 2/2418; A61F 2/2436

(57) ABSTRACT

Apparatus and methods are described for treating a subject with a diseased valve (24), and for use with a delivery device (34). A prosthetic valve apparatus (20) includes a valve support frame (30) and prosthetic valve leaflets (32). Prosthetic valve apparatus (20) is delivered to an aperture (22) through a first leaflet (25) of the diseased valve, while maintained in a generally cylindrical shape inside the delivery device. Subsequent to the delivery device being retracted, the support frame of the prosthetic valve apparatus becomes anchored to the first leaflet of the valve, by end portions (36A, 36V) at each end of the valve support frame at least partially inverting to trap tissue of the first leaflet between the inverted portions, and a central portion (38) of the valve support frame radially expanding. Other applications are also described.

### CROSS-REFERENCE TO RELATED APPLICATIONS

The present application claims priority from U.S. Provisional Application 62/476,979 to Benichou, entitled “Invertible valve support frame for use with prosthetic heart valve apparatus,” filed Mar. 27, 2017, which is incorporated herein by reference.

The present application is related to a PCT application being filed on even date herewith, entitled “Docking elements,” which claims priority from US Provisional Application 62/476,989 to Benichou, entitled “Docking element,” filed. Mar. 27, 2017. The aforementioned applications are incorporated herein by reference.

### FIELD OF EMBODIMENTS OF THE INVENTION

Some applications of the present invention generally relate to medical apparatus and methods. Specifically, some applications of the present invention relate to apparatus and methods for use with a mitral valve.

### BACKGROUND

Atrioventricular valves are cardiac valves that prevent backflow from the ventricles into the atria during systole. They are anchored to the wall of the heart at the fibrous skeleton by anchoring tendons named chordae tendineae. The chordae tendineae are attached to papillary muscles. Together, the papillary muscles and the chordae tendineae Keep the valves front prolapsing into the atria when they close during systole. The actual opening and closing of the valves is caused by a pressure gradient across the valve. The left-side atrioventricular valve is a bicuspid valve having two leaflets, and is commonly known as the mitral valve. The right-side atrioventricular valve is a tricuspid valve, having three leaflets. Both of these valves may be damaged and dysfunctional, resulting in leakage during systole, requiring the valves to be repaired or replaced.

While the mitral valve is generally an ellipse or D-shaped, the tricuspid valve is more circular. The left ventricle pumps oxygenated blood around the body and so the mitral valve has to withstand a higher pressure than the tricuspid valve, which only has to pump deoxygenated blood to the nearby lungs.

Occasionally, the mitral valve is congenitally abnormal or destroyed by infection or a bacterial endocarditis. More often, the mitral valve becomes degenerative with age, or as a result of rheumatic fever. There are different valvular heart disorders associated with the mitral valve such as mitral stenosis and mitral regurgitation. In the case of mitral stenosis, the valve orifice, i.e., the cross-section available for blood passage is reduced because of calcium nodes, leaflet thickening and/or reduced leaflet mobility, and, consequently, the valve does not allow normal blood flow. To overcome the damaged valve and to transport the same amount of blood, the left atrium requires a higher pressure than normal. The constant pressure overload of the left atrium may cause it to increase in size and become more prone to develop atrial fibrillation and to lose the atrial kick. The loss of the atrial kick due to atrial fibrillation can cause a precipitous decrease in cardiac output. A reduction in cardiac output, associated with acceleration of heart rate and shortening of the diastolic time, frequently leads to congestive heart failure. In most cases, mitral stenosis is due to rheumatic heart disease. The treatment options for mitral stenosis include medical management, surgical repair, surgical replacement of the valve, and percutaneous balloon valvuloplasty.

Mitral regurgitation causes heart murmurs and may have severe physiological consequences. Mitral regurgitation is caused either by ischemic heart disease (such cases being called “ischemic mitral regurgitation”), or mitral valve prolapse. Ischemic mitral regurgitation is a result of ventricular remodeling which is secondary to ischemic heart disease. The heart's posterior wall, which is not attached to the heart's fibrous skeleton, dilates. As a result of the change of the left ventricular geometry, the posterior leaflet, which is attached to the posterior heart wall, is displaced and misaligned from the anterior leaflet which results in mitral regurgitation.

Mitral valve prolapse is a condition caused by degeneration of the valve's connective tissue. Patients with classic mitral valve prolapse have surplus connective tissue. This weakens the leaflets and adjacent tissue, resulting in increased leaflet area and elongation of the chordae tendineae. Elongation of the chordae tendineae often causes rupture. Tweaked leaflets may be displaced in some portion of one or both of the abnormally thickened mitral valve leaflets into the left atrium during systole. Advanced lesions lead to leaflet folding, inversion, and displacement toward the left atrium. The abnormal leaflet structure leads to incomplete closure of the mitral valve and consequent mitral regurgitation.

In mitral regurgitation, the heart has to work harder by pumping not only the regular volume of blood, but also the extra volume of blood that is regurgitated back into the left atrium. The added workload creates an excessive strain on the left ventricle, which can lead to heart failure.

While patients with mild to moderate mitral regurgitation caused by mitral valve prolapse might experience no symptoms, increasing severity, even without symptoms, increases the load on the left ventricle. Over time, this can result in ventricular dilatation and congestive heart failure.

Mitral valve disease is conventionally treated by open heart surgery; either by surgical repair, which is usually performed using an annuloplasty ring, or by surgical replacement with a valve prosthesis. In some cases, such as when the valve is too damaged, mitral valves may require replacement. Mitral valve replacement may be performed robotically or manually. Surgical valve replacement or repair is often a demanding operation as it requires cardiopulmonary bypass and it can expose patients, especially elderly ones, to many risks.

A large variety of percutaneous or transcutaneous medical procedures are currently being developed and/or practiced. For example, transcatheter procedures are known for replacement of aortic and pulmonary heart valves. These procedures, which are performed under local anesthesia in the cardiac catheterization lab, rather than by cardiac surgery, offer benefits to these patients. According to such approaches, the valve is inserted on a delivery device similar to a catheter or a sheath and then implanted in the desired location via access through a large blood vessel such as the femoral artery, for example. This involves making a very small perforation in the patient's skin, such as in the groin area, in order to access the femoral artery. This minimally invasive option is usually safer than open heart surgery, and recovery times are typically shorter.

### SUMMARY OF EMBODIMENTS

In accordance with some applications of the present invention, an aperture is created in one of the leaflets of a subject's diseased mitral valve, e.g., using techniques described in U.S. Pat. No. 8,408,214 to Spenser, and/or U.S. Pat. No. 9,326,852 to Spenser, both of which patents are incorporated herein by reference. For example, an aperture may be created in mitral valve by piercing the anterior mitral leaflet away from the mating edges of the anterior and posterior leaflets and, typically, near the center of anterior leaflet. For some applications, a prosthetic valve apparatus is radially expanded within the aperture, as described in further detail hereinbelow, thereby forcing the edges of the leaflets together. For some applications, the prosthetic valve apparatus is thereby configured to close and void the native orifice of mitral valve.

Typically, the prosthetic valve apparatus includes a valve support frame and prosthetic valve leaflets coupled to an inner surface of the valve support frame. Typically, the valve support frame is inserted into the aperture, by a delivery device (e.g., a catheter, or a sheath) being advanced and positioned through the aperture while the prosthetic valve apparatus is disposed inside the delivery device. Typically, while disposed inside the delivery device, the valve support frame is radially constrained by the delivery device. The valve support frame is typically configured to define a generally cylindrical shape while disposed in its radially-constrained configuration within the delivery device. For some applications, in order to crimp the valve support frame for placement inside the delivery device, techniques as described hereinbelow are used.

Subsequent to the delivery device being advanced and positioned through the aperture, the delivery device is typically retracted. Typically, the valve support frame is shape set (using techniques as described hereinbelow), such that upon being released by the delivery device (e.g., due to the retraction of the delivery device), (a) portions at each end of the support frame at least partially invert such as to trap tissue of the leaflet of the valve (e.g. the tissue surrounding the aperture) between the inverted portions, and (b) a central portion of the support frame radially expands. For some applications, the central portion is radially expanded by inflating a balloon inside the central portion, and, for some such applications, the portions at each end of the support frame are prevented from inverting until the central portion has been expanded.

An atrial end portion is configured to invert such as to contact tissue of the atrial side of the valve leaflet, a ventricular end portion is configured to invert such as to contact tissue of the ventricular side of the valve leaflet, and the central portion is configured to radially expand against tissue of the leaflet that defines the aperture. Typically, the valve support frame is configured to become anchored to the leaflet of the valve, by virtue of (a) the atrial and ventricular end portions inverting such as to trap tissue of the leaflet of the valve between the inverted portions, and (b) the central portion of the support frame radially expanding.

There is therefore provided, in accordance with some applications of the present invention, apparatus for treating a subject with a diseased valve, and for use with a delivery device, the apparatus including:

a prosthetic valve apparatus including a valve support frame and prosthetic valve leaflets coupled to an inner surface of the valve support frame,

the prosthetic valve apparatus being configured:
to be delivered to an aperture through a first leaflet of the diseased valve, while disposed inside the delivery device, the valve support frame, while disposed within the delivery device, being configured to be maintained in a radially-constrained configuration, in which the valve support frame of the prosthetic valve apparatus is generally cylindrically shaped, and
   such that, subsequent to the delivery device causing being retracted. the support frame of the prosthetic valve apparatus becomes anchored to the first leaflet of the valve, by:
      end portions at each end of the valve support frame at least partially inverting such as to trap tissue of the first leaflet of the valve between the inverted portions, and
      a central portion of the valve support frame radially expanding.

In some applications, the diseased valve includes an annulus that defines a valve annulus diameter, and the central portion of the valve support frame is configured to radially expand such that a ratio of (a) an inner diameter of the valve support frame at a location at which the prosthetic valve leaflets are coupled to the inner surface of the valve support frame to (b) the valve annulus diameter is less than 5:6.

In some applications, the apparatus further includes first, second and third portions of a material,

the first and second portions of the material being coupled to respective end portions, such that when the end portions at least partially invert, the first and second portions of the material are disposed between the end portions and the tissue of the first leaflet of the diseased valve, and
the third portion of the material being coupled to an inner surface of the central portion of the valve support frame.

In some applications, at least one of the end portions is configured to at least partially invert, such that a portion of the end portion defines a tangent thereto that is perpendicular to a longitudinal axis of the valve support frame.

In some applications, the central portion of the valve support frame of the prosthetic valve apparatus is configured to radially expand, a ratio of (a) an outer diameter of the valve support frame when the central portion is radially expanded, to (b) an outer diameter of the valve support frame when in its radially-constrained configuration being greater than 4:1.

In some applications, the central portion of the valve support frame of the prosthetic valve apparatus is configured to radially expand such that a ratio between an outer diameter of the valve support frame and a minimum inner diameter of the valve support frame is less than 3:2.

In some applications:

the end portions at each end of the valve support frame are configured to at least partially invert automatically, in response to the valve support frame being released from the delivery device; and

the central portion of the support frame is configured to radially expand automatically, in response to the valve support frame being released from the delivery device.

In some applications, the apparatus is for use with a balloon, and the central portion of the support frame is configured to radially expand by the balloon being inflated inside the central portion.

In some applications, the apparatus further includes elements configured to prevent the end portions from at least partially inverting until the balloon has been inflated inside the central portion of the valve support frame.

There is further provided, in accordance with some applications of the present invention, a method of treating a subject with a diseased valve, including:

creating an aperture through a first leaflet of the diseased valve;

advancing and positioning, through the aperture, a delivery device, a prosthetic valve apparatus being disposed within the delivery device,
the prosthetic valve apparatus including a valve support frame, and prosthetic valve leaflets coupled to an inner surface of the valve support frame,
the valve support frame, while disposed within the delivery device, being maintained in a radially-constrained configuration, in which the valve support frame is generally cylindrically shaped; and

subsequently, causing the valve support frame of the prosthetic valve apparatus to become anchored to the first leaflet of the diseased valve, by:
causing end portions at each end of the valve support frame to at least partially invert, such as to trap tissue of the first leaflet of the diseased valve between the inverted portions, and
causing a central portion of the valve support frame to radially expand.

In some applications, the diseased valve includes an annulus that defines a valve annulus diameter, and causing the central portion of the valve support frame to radially expand includes causing the valve support frame to assume a configuration in which a ratio of (a) an inner diameter of the valve support frame at a location at which the prosthetic valve leaflets are coupled to the inner surface of the valve support frame to (b) the valve annulus diameter is less than 5:6.

In some applications, the valve support frame includes a material, first and second portions of which are coupled to respective end portions, and a third portion of which is coupled to an inner surface of the central portion of the valve support frame, and causing end portions at each end of the valve support frame to at least partially invert includes bringing the first and second portions of the material into contact with the tissue of the first leaflet of the diseased valve, such that the first and second portions of the material are disposed between the respective end portions and the tissue of the first leaflet of the diseased valve.

In some applications, causing the end portions to at least partially invert includes causing at least one of the end portions to invert, such that a portion of the end portion defines a tangent thereto that is perpendicular to a longitudinal axis of the valve support frame.

In some applications, causing the central portion of the valve support frame to radially expand includes causing the central portion of the valve support frame to radially expand, a ratio of (a) an outer diameter of the valve support frame when the central portion of the valve support frame is radially expanded, to (b) an outer diameter of the valve support frame when in its radially-constrained configuration being greater than 4:1.

In some applications, causing the central portion of the valve support frame to radially expand includes causing the valve support frame to assume a configuration in which a ratio between an outer diameter of the valve support frame and a minimum inner diameter of the valve support frame is less than 3:2.

In some applications:

causing portions at each end of the support frame to at least partially invert includes automatically causing the portions at each end of the support frame to at least partially invert by releasing the valve support frame from the delivery device; and

causing the central portion of the support frame to radially expand includes automatically causing the central portion of the support frame to radially expand by releasing the valve support frame from the delivery device.

In some applications, causing the central portion of the valve support frame to radially expand includes at least partially radially expanding the central portion of the valve support frame by inflating a balloon inside the central portion.

In some applications, the method further includes preventing the portions at each end of the valve support frame from at least partially inverting until the balloon has been inflated inside the central portion of the valve support frame.

The present invention will be more fully understood from the following detailed description of embodiments thereof, taken together with the drawings, in which:

### BRIEF DESCRIPTION OF THE DRAWINGS

FIGS. 1A and 1B are schematic illustrations of respective stages of the implantation of a prosthetic valve apparatus within an aperture in a leaflet of a diseased valve, in accordance with some applications of the present invention;

FIG. 2 is a schematic illustration of a support frame of the prosthetic valve apparatus when disposed in a radially-constrained configuration thereof, in accordance with some applications of the present invention;

FIGS. 3A and 3B are schematic illustrations of a support frame of the prosthetic valve apparatus when disposed in a non-radially constrained configuration thereof, in accordance with some applications of the present invention;

FIG. 4 is a schematic illustration of a support frame of the prosthetic valve apparatus when disposed in a non-radially-constrained configuration thereof, in accordance with some alternative applications of the present invention;

FIG. 5 is a schematic illustration of the support frame of the prosthetic valve apparatus when disposed in a non-constrained configuration thereof, portions of the support frame having a material coupled thereto, in accordance with some applications of the present invention; and

FIG. 6 is a schematic illustration of the support frame of the prosthetic valve apparatus being crimped, in accordance with some applications of the present invention.

### DETAILED DESCRIPTION OF EMBODIMENTS

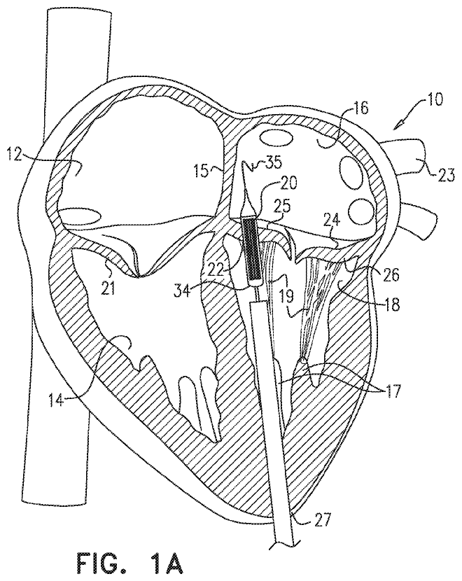

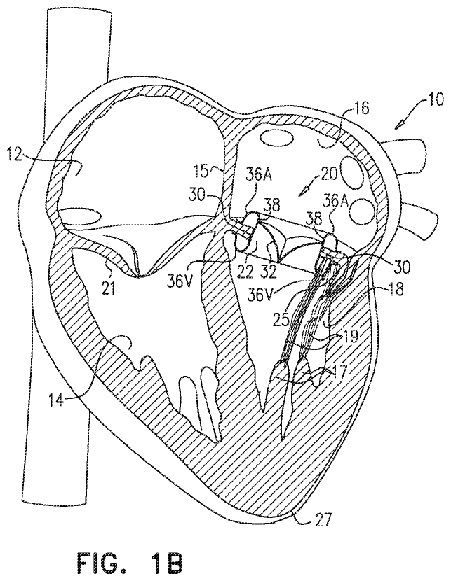

Reference is now made to FIGS. 1A and 1B, which are schematic illustrations of respective stages of the implantation of a prosthetic valve apparatus 20 within an aperture 22 in a leaflet of a diseased mitral valve 24, in accordance with some applications of the present invention. In FIGS. 1A and 1B, a vertical section through a heart is shown. The heart 10 consists of a right atrium 12, a right ventricle 14, a left atrium 16 and a left ventricle 18. The right and left atria 12, 16 are separated by the interatrial septum 15. The right atrium 12 and right ventricle 14 are separated by a tricuspid valve 21, and the left atrium 16 and left ventricle 18 are separated by a bicuspid valve, known as the mitral valve 24. The mitral valve consists of an anterior leaflet 25 and a posterior leaflet 26 having edges that separate, during diastole, as blood flows from the left atrium into the left ventricle, and subsequently come together, during systole, as the left ventricle 18 dilates and contracts to force blood into the aorta (not shown).

Deoxygenated blood from the body flows through the vena cava (not shown) into the right atrium 12 and flows into the right ventricle 14 through the tricuspid valve 21 as it dilates. The right ventricle 14 pumps the blood via the pulmonary artery (not shown) to the lungs. Oxygenated blood from the lungs flows via the pulmonary veins 23 into the left atrium 16 and fills the left ventricle 18 via the orifice between the edges of the leaflets 25, 26 of the mitral valve 24. Systole of the heart 10 pumps the oxygenated blood through the aorta (not shown) and around the body. The anterior and posterior leaflets 25, 26 of the mitral valve 24 flex together as the left ventricle 18 contracts, to prevent blood being pushed back to the left atrium 16. Papillary muscles 17, chordae tendineae 19 and the heart apex 27 are also shown. Some applications of the present invention are directed toward apparatus and method for treating a patient with a dysfunctional or diseased mitral valve 24. Alternatively or additionally, the apparatus and methods described herein are used to treat other diseased valves, such as the tricuspid valve, mutatis mutandis.

For some applications, an aperture 22 is created in one of the leaflets 25, 26, e.g., using techniques described in U.S. Pat. No. 8,408,214 to Spenser, and/or U.S. Pat. No. 9,326,852 to Spenser, both of which are incorporated herein by reference. For example, aperture 22 may be created in mitral valve 24 by piercing anterior mitral leaflet 25 away from the mating edges of leaflets 25, 26 and, typically, near the center of anterior leaflet 25. For some applications, prosthetic valve apparatus 20 is radially expanded within the aperture (e.g., by self-expanding, or by being expanded by a balloon), as described in further detail hereinbelow, thereby forcing the edges of leaflets 25, 26 together. For some applications, the prosthetic valve apparatus is thereby configured to close and void the native orifice of mitral valve 24. For some applications (not shown), the aperture is created in posterior leaflet 26, and additional steps of the procedure are performed with respect to the posterior leaflet, mutatis mutandis.

As described in U.S. Pat. No. 8,408,214 to Spenser, and/or U.S. Pat. No. 9,326,852 to Spenser, both of which are incorporated herein by reference, access to mitral valve 24 may be via the left atrium 16 or via the left ventricle 18, and there are a number of possible percutaneous routes. For example, as shown in FIGS. 1A and 1B, access to the valve may be transapical i.e. via the heart apex 27. Alternatively (not shown), access to the left ventricle 18 is achieved via a transvascular approach, typically using transvascular catheterization. For example, transaortic access may be used. Further alternatively, the mitral valve is accessed via right atrium 12, e.g., by a transvascular approach, Typically, the transvascular approach uses transvascular catheterization, with right atrium 12 being accessed via the vena cava and left atrium 16 being accessed by piercing interatrial septum 15.

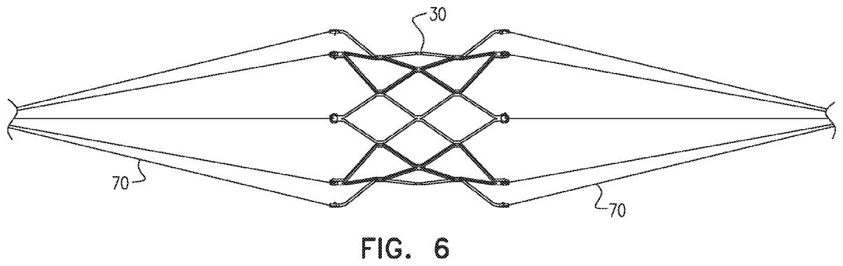

As shown in FIG. 1B, typically prosthetic valve apparatus 20 includes a valve support frame 30 and prosthetic valve leaflets 32 coupled to the inner surface of the valve support frame. Typically, the support frame is inserted into aperture 22, by a delivery device 34 (e.g., a catheter, or a sheath) being advanced and positioned through the aperture while the prosthetic valve apparatus is disposed inside the delivery device, e.g., as shown in FIG. 1A. For some applications, advancement of the delivery device is guided by a guidewire 35. Typically, while disposed inside the delivery device, the valve support frame is maintained in a radially constrained configuration by the delivery device. The valve support frame is typically configured to define a generally cylindrical shape while disposed in its radially-constrained configuration within the delivery device. For some applications, in order to crimp the valve support frame (i.e., in order to axially elongate and radially constrict the valve support frame) for placement inside the delivery device, techniques as described hereinbelow with reference to FIG. 6 are used.

Subsequent to delivery device 34 being advanced and positioned through aperture 22, the delivery device is typically retracted. Typically, the valve support frame is shape set (using techniques as described hereinbelow), such that upon being released by the delivery device (due to the retraction of the delivery device), (a) atrial and ventricular portions 36A and 36V at respective ends of the support frame at least partially invert such as to trap tissue of the leaflet of the valve (e.g. the tissue surrounding the aperture) between the inverted portions, and (b) a central portion 38 of the support frame radially expands. Typically, the valve support frame is configured to thereby become anchored to the leaflet of the valve. Typically, portion 36A is configured to invert such as to contact tissue of the atrial side of the valve leaflet, portion 36V is configured to invert such as to contact tissue of the ventricular side of the valve leaflet, and central portion 38 is configured to radially expand against tissue of the leaflet that defines the aperture.

Typically, by implanting prosthetic valve apparatus 20 inside aperture 22, most of the tissue of anterior leaflet 25 is pushed toward posterior leaflet 26, e.g., as shown in FIG. 1B. Typically, in this manner, the native mitral valve orifice is sealed. In addition, typically, the left ventricular outflow tract is cleared of the tissue of the native mitral valve leaflets, by the valve support frame trapping the tissue of the native mitral valve leaflets, and/or by pushing the tissue radially outwards.

Although some applications of the present invention are described in which valve support frame 30 is self-expandable, the scope of the present invention includes applications in which some of the shape changes of the valve support frame described herein are performed at least partially manually, mutatis mutandis. For example, for some applications, the valve support frame is configured such that when central portion 38 of the valve support frame is released from delivery device 34, the central portion expands to a larger diameter but is still partially constrained by the tissue surrounding the central portion. Typically, for such applications, a balloon (not shown) is inflated inside the central portion of the valve support frame such as to radially expand the central portion, and thereby push tissue of the native valve leaflets radially outwards. For some such applications, prior to the balloon expansion of the central portion of the valve support frame, the atrial and ventricular end portions are prevented from inverting (e.g., by using a clip, or a similar element, to clip the end portions). Subsequent to the balloon expansion of the central portion of the valve support frame, the atrial and ventricular end portion are allowed to invert (e.g., by unclipping the end portions). As described hereinabove, the inversion of the atrial and ventricular end portions typically traps tissue of the leaflet of the valve (e.g. the tissue surrounding the aperture) between the inverted portions.

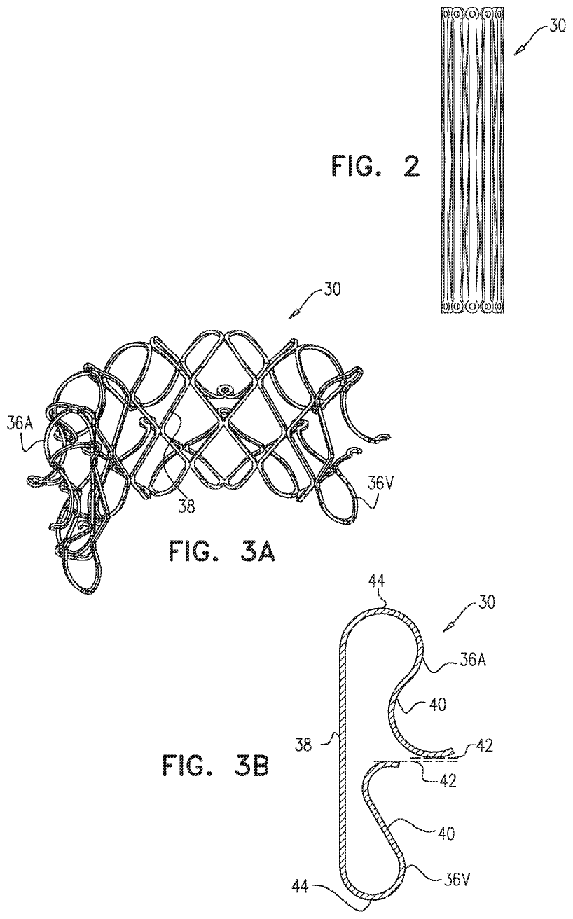

Reference is now made to FIG. 2, which is a schematic illustration of valve support frame 30, the valve support frame being shaped in its radially-constrained configuration, in accordance with some applications of the present invention. Reference is also made to FIGS. 3A and 3B, which are schematic illustrations of valve support frame 30 of the prosthetic valve apparatus when disposed in a non-constrained configuration thereof, in accordance with some applications of the present invention. FIG. 3A shows a three-dimensional schematic illustration of the support frame, and FIG. 3B shows a profile view of a section of the support frame. As shown in FIGS. 3A and 3B, typically, the valve support frame is shape set such that in its non-constrained configuration the end portions 36A and 36V become inverted relative to the radially-constrained configuration (i.e., surfaces of the portion that were previously inner surfaces of the cylinder, become outer surfaces of the valve support frame), and central portion 38 becomes radially expanded.

For some applications (not shown), in its non-radially-constrained configuration, central portion 38 of valve support frame 30 has a frustoconical shape, the central portion converging in the direction running from the atrial end portion 36A to ventricular end portion 36V. For some applications, at least a portion 40 of end portion 36A and/or end portion 36V inverts by more than 180 degrees. Typically, when the valve support frame is in the non-radially-constrained configuration, shapes of end portions 36A and 36V are different from one another, in order to conform with the shapes of, respectively, the surrounding atrial tissue, and the surrounding ventricular tissue. For some applications, when the valve support frame is in the non-radially-constrained configuration, the frame has a relatively high radial stiffness, (a) since by virtue of the inverted portion, the valve support frame defines a double cylinder at the ends, and/or (b) due to bent portions 44 (which are disposed at the tips of the support frame when the support frame is in the non-radially-constrained configuration) contributing to the stiffness of the valve support frame. Alternatively or additionally, when the valve support frame is in the non-radially-constrained configuration, the frame has a relatively high fatigue resistance, (a) since by virtue of the inverted portion, the valve support frame defines a double cylinder at the ends, and/or (b) due to bent portions 44 (which are disposed at the tips of the support frame when the support frame is in the non-radially-constrained configuration) contributing to the fatigue resistance of the valve support frame.

For some applications, the end of end portion 36A and/or end portion 36V curves radially outwardly, such that a portion of end portion 36A and/or end portion 36V defines a tangent 42 thereto that is perpendicular to a longitudinal axis 46 (FIG. 5) of the valve support frame.

Typically, a ratio of an outer diameter Do (shown in FIG. 5) of valve support frame 30, when the valve support frame is in its non-radially-constrained configuration, to an outer diameter of the valve support frame 30, when the valve support frame is in its radially-constrained configuration is greater than 4:1, e.g., greater than 8:1. For some applications, the outer diameter of valve support frame 30, when the valve support frame is in its non-radially-constrained configuration is greater than 40 mm (e.g., greater than 50 mm) and/or less than 70 mm (e.g., less than 60 mm), e.g., between 40 and 70 mm, or between 50 and 60 mm. For some applications, the outer diameter of valve support frame 30, when the valve support frame is in its radially-constrained configuration is greater than 5 mm (e.g., greater than 6 mm) and/or less than 10 mm (e.g., less than 8 mm), e.g., between 5 and 10 mm, or between 6 and 8 mm.

It is noted that, for some applications, the dimensions described herein as being the dimensions of the valve support frame, when in its non-radially-constrained configuration, are the dimensions that the valve support frame assumes when no forces are being applied to the valve support frame. Typically, when the valve support frame is implanted inside the aperture within the subject's mitral valve leaflet, there are some forces that are exerted upon the valve support frame by the subjects tissue. Therefore, for some applications, the configuration that the valve support frame assumes when implanted inside the aperture within the subject's mitral valve leaflet is slightly different from the non-radially-constrained configuration of the valve support frame. However, the configuration that the valve support frame assumes when implanted inside the aperture within the subject's mitral valve leaflet is typically substantially similar to the non-radially constrained configuration of the valve support frame. Therefore, the configuration that the valve support frame assumes when implanted inside the aperture within the subject's mitral valve leaflet is described herein in the specification and the claims as being a substantially non-radially-constrained configuration of the valve support frame.

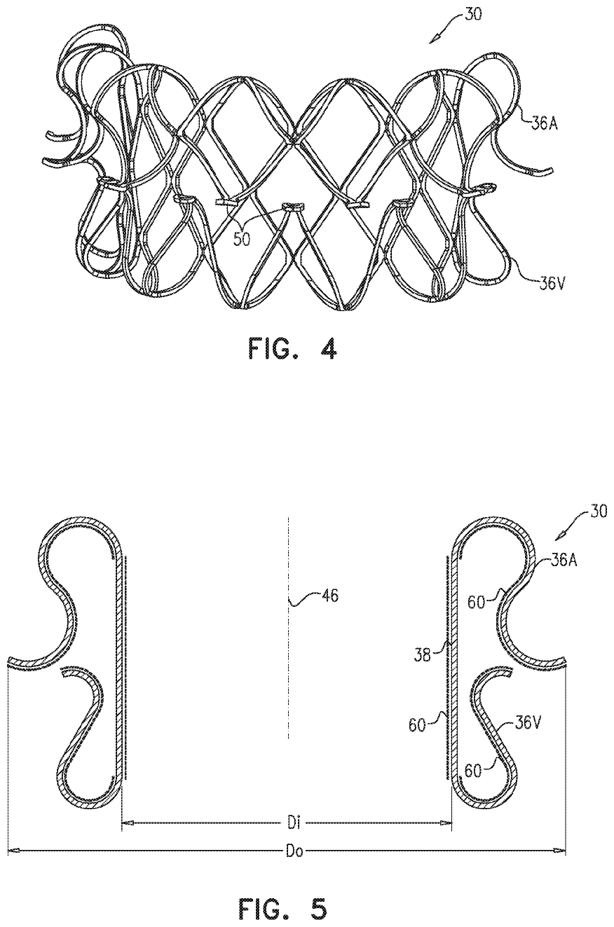

Reference is now made to FIG. 4, which is a schematic illustration of support frame 30 of the prosthetic valve apparatus 20 when disposed in a non-constrained configuration thereof, in accordance with some alternative applications of the present invention. In the frame shown in FIG. 4, when the valve support frame is in its non-radially-constrained configuration, junctions 50 of struts of inverted atrial end portion 36A at the end of inverted atrial end portion 36A alternate with junctions 50 of struts of inverted ventricular end portion 36V at the end of inverted ventricular end portion 36V. In all other respects, valve support frame 30 as shown in FIG. 4 is generally similar to the valve support frame shown in FIGS. 3A and 3B.

FIG. 5 is a schematic illustration of a cross-sectional view of support frame 30 of prosthetic valve apparatus 20 when disposed in a non-radially-constrained configuration thereof, portions of end portions of the support frame that contact tissue of the valve leaflet having a material 60 coupled thereto, in accordance with some applications of the present invention. As described hereinabove, support frame 30 is configured to become anchored to the valve leaflet by portion 36A inverting such as to contact tissue of the atrial side of the valve leaflet, portion 36V inverting such as to contact tissue of the ventricular side of the valve leaflet, and central portion 38 radially expanding against tissue of the leaflet that defines the aperture. For some applications, with respect to the inverting portions, first and second portions of material 60 are coupled to the side of the portion that is configured to contact tissue of the valve leaflet (typically, in order to facilitate tissue in-growth). Thus, material 60 is disposed on what was the outer surfaces of the inverting portions when the valve support frame was in its non-radially constrained configuration. With respect to central portion 38, a third portion of the material is disposed on what was (and remains) the inner surface, typically in order to provide sealing of prosthetic valve leaflets 32 (FIG. 1B), with respect to the valve support frame. Typically, material 60 includes PET, PTFE, nylon, and/or pericardial tissue.

With reference to FIG. 5, for some applications, when the valve support frame 30 is in the non-radially-constrained configuration, a ratio between an outer diameter Do of the support frame and a minimum inner diameter Di of the support frame is less than 3:2, e.g., less than 5:4.

For some applications, when the valve support frame is in its non-radially-constrained configuration the inner diameter of valve support frame 30 at a location at which the prosthetic valve leaflets are coupled to the inner surface of the valve support frame, is less than the measured diameter of the native mitral valve annulus (the diameter of the native mitral valve annulus typically being measured using a mitral measuring ring, and/or using imaging methods, such as ultrasound). For example, the ratio of the inner diameter of valve support frame 30 to the diameter of the native mitral valve annulus may be less than 5:6. Thus, for example, if the diameter of the native mitral valve annulus as measured by a mitral valve measuring ring is 30 mm, the inner diameter of the valve support frame may be less than 25 mm.

Since the prosthetic valve leaflets 32 are coupled to the inside of the valve support frame, the diameter of the prosthetic valve leaflets is typically defined by the inner diameter of the valve support frame. Thus, for some applications, the diameter of the prosthetic valve leaflets is less than that of the native mitral valve annulus. For some applications, one or more advantages of the prosthetic valve leaflets having a diameter that is less than that of the native mitral valve annulus, relative to a prosthetic valve apparatus having prosthetic valve leaflets that have a greater diameter, may include: the prosthetic valve apparatus having a lower crimp profile, there being less foreign matter inside the subject's heart, lower forces being exerted on the valve leaflets, better anchoring of the prosthetic valve apparatus, less interference with the native anatomy, and/or better preservation of a clear left ventricular outflow tract.

Reference is now made to FIG. 6, which is a schematic illustration showing a stage of the crimping process of valve support frame 30, in accordance with some applications of the present invention. For some applications, in order to place the support frame in delivery device 34 (FIG. 1A), the valve support frame is crimped (i.e., the valve support frame is axially elongated and radially constricted). For some such applications, a plurality of elongate elements 70 (e.g., strings or wire) are coupled to the end of the valve support frame, and the ends are pulled away from one another by means of the elongate elements.

Typically, valve support frame 30 is made from a super elastic, shape memory material, such as nitinol alloy, which is shape set to define the non-radially constrained shape of the valve support element. Therefore, in response to being released from delivery device 34 (FIG. 1A), the device automatically reverts to its non-radially-constrained configuration. Alternatively or additionally, the valve support frame is configured to undergo a phase change as it approaches body temperature. Further alternatively or additionally, at least some of the shape changes that the valve support frame undergoes are performed manually, e.g., as described hereinabove.

For some applications, valve support frame 30 is at least partially treated with at least one therapeutic agent. Optionally, the therapeutic agent is configured to be eluted into the cardiac tissue or into the cardiac chamber over time. For example, a therapeutic agent may be used that is known to significantly reduce or even prevent a variety of pathological conditions including, but not limited to, arrhythmias, thrombosis, stenosis and inflammation. Accordingly, the therapeutic agent may include at least one of an anti-arrhythmic agent, anticoagulant, an antioxidant, a fibrinolytic, a steroid, an anti-apoptotic agent, and/or an anti-inflammatory agent. Alternatively or additionally, the therapeutic agent may be capable of treating or preventing other disease or disease processes such as microbial infections and heart failure. In these instances, the therapeutic agent may include an inotropic agent, a chronotropic agent, an anti-microbial agent, and/or a biological agent such as a cell or protein.

For some applications, prosthetic valve leaflets 32 (FIG. 1B), which are mounted on valve support frame 30, are made from one or more pieces of biological material formed into a valve. The valve leaflets are configured to act as a one-way valve, whereby in their open positions with respect to one another the leaflets allow flow to pass through the prosthetic valve apparatus from the inlet (on the atrial side) to the outlet (on the ventricular side), whereas a reverse flow is prevented due to collapsible slack portions of the valve leaflets that collapse inwardly to block the reverse flow.

For some applications, materials of biological origin (e.g., bovine, porcine, equine, ovis aries pericardial tissue) are used for prosthetic valve leaflets 32 (FIG. 1B), which are mounted on valve support frame 30. The prosthetic valve leaflets are typically operatively secured to the valve support frame 30, such as by sutures. Alternatively, the prosthetic valve leaflets may be secured to the valve support frame 30 in a variety of different manners including, for example, using clips, pins, staples, and the like.

For some applications, the apparatus and methods described herein are performed with respect to a tricuspid valve, and/or a different valve in a subject's body, mutatis mutandis.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for treating a subject with a diseased valve, and for use with a delivery device, the apparatus comprising:
a prosthetic valve apparatus comprising a valve support frame and prosthetic valve leaflets coupled to an inner surface of the valve support frame,
the prosthetic valve apparatus being configured:
to be delivered to an aperture through a first leaflet of the diseased valve, while disposed inside the delivery device, the valve support frame, while disposed within the delivery device, being configured to be maintained in a radially-constrained configuration, in which the valve support frame of the prosthetic valve apparatus is generally cylindrically shaped, and
such that, subsequent to the delivery device causing being retracted, the support frame of the prosthetic valve apparatus becomes anchored to the first leaflet of the valve, by:
end portions at each end of the valve support frame at least partially inverting such as to trap tissue of the first leaflet of the valve between the inverted portions, and
a central portion of the valve support frame radially expanding.

2. The apparatus according to claim 1, wherein the diseased valve includes an annulus that defines a valve annulus diameter, and the central portion of the valve support frame is configured to radially expand such that a ratio of (a) an inner diameter of the valve support frame at a location at which the prosthetic valve leaflets are coupled to the inner surface of the valve support frame to (b) the valve annulus diameter is less than 5:6.

3. The apparatus according to claim 1, further comprising first, second and third portions of a material,
the first and second portions of the material being coupled to respective end portions, such that when the end portions at least partially invert, the first and second portions of the material are disposed between the end portions and the tissue of the first leaflet of the diseased valve, and
the third portion of the material being coupled to an inner surface of the central portion of the valve support frame.

4. The apparatus according to claim 1, wherein at least one of the end portions is configured to at least partially invert, such that a portion of the end portion defines a tangent thereto that is perpendicular to a longitudinal axis of the valve support frame.

5. The apparatus according to claim 1, wherein the central portion of the valve support frame of the prosthetic valve apparatus is configured to radially expand, a ratio of (a) an outer diameter of the valve support frame when the central portion is radially expanded, to (b) an outer diameter of the valve support frame when in its radially-constrained configuration being greater than 4:1.

6. The apparatus according to claim 1, wherein the central portion of the valve support frame of the prosthetic valve apparatus is configured to radially expand such that a ratio between an outer diameter of the valve support frame and a minimum inner diameter of the valve support frame is less than 3:2.

7. The apparatus according to claim 1, wherein:
the end portions at each end of the valve support frame are configured to at least partially invert automatically, in response to the valve support frame being released from the delivery device; and
the central portion of the support frame is configured to radially expand automatically, in response to the valve support frame being released from the delivery device.

8. The apparatus according to claim 1, wherein the apparatus is for use with a balloon, and wherein the central portion of the support frame is configured to radially expand by the balloon being inflated inside the central portion.

9. The apparatus according to claim 8, further comprising elements configured to prevent the end portions from at least partially inverting until the balloon has been inflated inside the central portion of the valve support frame.

10. A method of treating a subject with a diseased valve, comprising:
creating an aperture through a first leaflet of the diseased valve;
advancing and positioning, through the aperture, a delivery device, a prosthetic valve apparatus being disposed within the delivery device,
the prosthetic valve apparatus including a valve support frame, and prosthetic valve leaflets coupled to an inner surface of the valve support frame,
the valve support frame, while disposed within the delivery device, being maintained in a radially-constrained configuration, in which the valve support frame is generally cylindrically shaped; and
subsequently, causing the valve support frame of the prosthetic valve apparatus to become anchored to the first leaflet of the diseased valve, by:
causing end portions at each end of the valve support frame to at least partially invert, such as to trap tissue of the first leaflet of the diseased valve between the inverted portions, and
causing a central portion of the valve support frame to radially expand.

11. The method according to claim 10, wherein the diseased valve includes an annulus that defines a valve annulus diameter, and wherein causing the central portion of the valve support frame to radially expand comprises causing the valve support frame to assume a configuration in which a ratio of (a) an inner diameter of the valve support frame at a location at which the prosthetic valve leaflets are coupled to the inner surface of the valve support frame to (b) the valve annulus diameter is less than 5:6.

12. The method according to claim 10, wherein the valve support frame includes a material, first and second portions of which are coupled to respective end portions, and a third portion of which is coupled to an inner surface of the central portion of the valve support frame, and wherein causing end portions at each end of the valve support frame to at least partially invert comprises bringing the first and second portions of the material into contact with the tissue of the first leaflet of the diseased valve, such that the first and second portions of the material are disposed between the respective end portions and the tissue of the first leaflet of the diseased valve.

13. The method according to claim 10, wherein causing the end portions to at least partially invert comprises causing at least one of the end portions to invert, such that a portion of the end portion defines a tangent thereto that is perpendicular to a longitudinal axis of the valve support frame.

14. The method according to claim 10, wherein causing the central portion of the valve support frame to radially expand comprises causing the central portion of the valve support frame to radially expand, a ratio of (a) an outer diameter of the valve support frame when the central portion of the valve support frame is radially expanded, to (b) an outer diameter of the valve support frame when in its radially-constrained configuration being greater than 4:1.

15. The method according to claim 10, wherein causing the central portion of the valve support frame to radially expand comprises causing the valve support frame to assume a configuration in which a ratio between an outer diameter of the valve support frame and a minimum inner diameter of the valve support frame is less than 3:2.

16. The method according to claim 10, wherein:
causing portions at each end of the support frame to at least partially invert comprises automatically causing the portions at each end of the support frame to at least partially invert by releasing the valve support frame from the delivery device; and
causing the central portion of the support frame to radially expand comprises automatically causing the central portion of the support frame to radially expand by releasing the valve support frame from the delivery device.

17. The method according to claim 10, wherein causing the central portion of the valve support frame to radially expand comprises at least partially radially expanding the central portion of the valve support frame by inflating a balloon inside the central portion.

18. The method according to claim 17, further comprising preventing the portions at each end of the valve support frame from at least partially inverting until the balloon has been inflated inside the central portion of the valve support frame.
